(19) United States
(12) Patent Application Publication
(10) Pub. No.: US 2010/0267055 A1
(43) Pub. Date: Oct. 21, 2010

(54) ONE-STEP IMMUNOASSAYS EXHIBITING INCREASED SENSITIVITY AND SPECIFICITY

(75) Inventor: JOHN G. KONRATH, Lake Villa, IL (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(73) Assignee: Abbott Laboratories, Abbott Park, IL (US)

(21) Appl. No.: 12/762,903

(22) Filed: Apr. 19, 2010

Related U.S. Application Data
(63) Continuation of application No. PCT/US2008/080634, filed on Oct. 21, 2008.
Application No. PCT/US2008/080634 is a continuation-in-part of application No. 12/199,909, filed on Aug. 28, 2008.
Application No. 12/199,909 is a continuation-in-part of application No. 11/875,908, filed on Oct. 21, 2007.

(51) Int. Cl. G01N 33/53

(52) U.S. Cl. 435/7.9; 436/501

(57) ABSTRACT

The present disclosure relates to immunoassays for detecting or quantifying at least one analyte of interest in a test sample which exhibits improved sensitivity or sensitivity and specificity compared to the immunoassay formats known in the art.

### RELATED APPLICATION INFORMATION

This application is a continuation application of PCT International Application PCT/US2008/080634 filed Oct. 21, 2008 (pending), is a continuation-in-part of application of U.S. application Ser. No. 12/199,909 filed Aug. 28, 2008 (pending), and is a continuation-in-part application of U.S. application Ser. No. 11/875,908, filed on Oct. 21, 2007 (abandoned), the contents of each of which are herein incorporated by reference.

### FIELD

The present disclosure generally relates to one-step immunoassays for detecting or quantifying at least one analyte of interest in a test sample. Specifically, in one aspect, the immunoassays of the present disclosure exhibit improved sensitivity by limiting non-specific background signal to levels below those that occur in conventional or traditional two-step immunoassay formats. In another aspect, the immunoassays of the present disclosure employ an optimized wash buffer or diluent and exhibit improved sensitivity and specificity compared to conventional or traditional two-step immunoassay formats known in the art.

### BACKGROUND

Immunoassays have proven to be particularly useful in testing for analytes of interest contained in test samples. In an immunoassay, the interaction of an analyte, such as an antigen, with a specific binding partner, such as an antibody, results in the formation of an analyte-binding partner complex. This complex can be detected by various measurements, such as, but not limited to, radioactivity, fluorescence, light absorption and light scattering. The results are then correlated with the presence or, absence, and ideally, with the concentration of the analyte in a test sample.

However, there still remains a need in the art for an immunoassay which enables an improved quantitative determination of analyte concentration having improved sensitivity in an accurate, yet simple manner.

### SUMMARY

The present disclosure relates to immunoassays. In one aspect, the present disclosure relates to one-step immunoassays that comprises the steps of:

a) incubating for a first incubation period a first mixture, the first mixture comprising (1) a test sample being assessed for at least one analyte of interest, (2) a first specific binding partner that is immobilized on a solid phase, wherein the first specific binding partner binds to the at least one analyte of interest, and (3) a second specific binding partner labeled with a first detectable label, wherein the analyte, first specific binding partner and second specific binding partner form a solid phase-first specific binding partner-analyte-second specific binding partner complex;

b) capturing the solid phase-first specific binding partner-analyte-second specific binding partner complex and isolating the solid phase-first specific binding partner-analyte-second specific binding partner complex from the supernatant of the first mixture;

c) removing freely accessible unbound second specific labeled binding partner from the captured solid phase first specific binding partner-analyte-second specific binding partner complex;

d) resuspending the captured solid phase-first specific binding partner-analyte-second specific binding partner complex to form a second mixture comprising resuspended solid phase-first specific binding partner-analyte-second specific binding partner complex;

e) incubating the second mixture for a second incubation period;

f) capturing the resuspended solid phase-first specific binding partner-analyte-second specific binding partner complex and isolating the solid phase-first specific binding partner-analyte-second specific binding partner complex from the supernatant of the second mixture;

g) removing residual unbound second specific labeled binding partner from the second mixture; and

h) detecting the labeled second specific binding partner from the first specific binding partner-analyte-second specific binding partner complex in step g) as a measure of the at least one analyte of interest.

In the immunoassay of the present disclosure, the freely-accessible unbound second specific labeled binding partner can be removed from the first mixture in step c) by washing.

In the above immunoassay, the residual unbound second specific labeled binding partner is removed from the second mixture in step g) by washing.

Additionally, in the above immunoassay, the second mixture of step d) can further comprise a third specific binding partner and the first mixture to form a second mixture, wherein said third specific binding partner is labeled with a second detectable label. The second detectable label may be identical to the first detectable label or different from the first detectable label. In one aspect, the second identical label is the same detectable label as the first detectable label.

In the above immunoassay, the first specific binding partner can be an antibody or an antigen. Specifically, if the first specific binding partner is an antibody, the antibody can be selected from the group consisting of a polyclonal antibody, a monoclonal antibody, a chimeric antibody, a human antibody, and an affinity maturated antibody.

In the above immunoassay, the solid phase can be selected from the group consisting of a magnetic particle, bead, test tube, microtiter plate, cuvette, membrane, a scaffolding molecule, film, filter paper, disc, and chip.

In the above immunoassay, the first detectable label and second detectable label can each be selected from the group consisting of a radioactive label, an enzymatic label, a chemiluminescent label, a fluorescent label, a thermometric label, and an immuno-polymerase chain reaction label. In one aspect of the present disclosure, the first detectable label and the second detectable label are the same. In another aspect of the present disclosure, the first detectable label and the second detectable label are different. In one aspect, the first detectable label and the second detectable label are the same.

In the above immunoassay, the second specific binding partner can be immobilized on a solid phase. If the second specific binding partner is immobilized on a solid phase, then the solid phase can be selected from the group consisting of a magnetic particle, bead, test tube, microtiter plate, cuvette, membrane, a scaffolding molecule, film, filter paper, disc, and chip.

In the above immunoassay, the second specific binding partner and the third specific binding partner can be the same or they can each be different. Moreover, in the above immunoassay, the second specific binding, the third specific binding partner or each of the second specific binding partner and the third specific binding partner can comprise multiple binding partners.

In the above immunoassay, the first incubation period comprises a period of from about 5 minutes to about 60 minutes. The second incubation period comprises a period of from about 30 seconds to about 30 minutes.

In the above immunoassay, the immunoassay relates the amount of said first specific binding partner-analyte-second specific binding partner complex in step g) to the amount of the at least one analyte of interest in the test sample either by use of a standard curve for the analyte, or by comparison to a reference standard. For example, in the above immunoassay, the amount of the at least one analyte of interest in the test sample is quantitated by measuring the amount of the second detectable label.

In a second aspect, as, the present disclosure relates to a one-step immunoassay that comprises the following steps:

a) incubating for a first incubation period a first mixture comprising: (1) a test sample being assessed for at least one analyte of interest, (2) a first specific binding partner that is immobilized on a solid phase, wherein the first specific binding partner binds to the at least one analyte of interest, and (3) a second specific binding partner labeled with a first detectable label, wherein the analyte, first specific binding partner and second specific binding partner form a solid phase-first specific binding partner-analyte-second specific binding partner complex;

b) capturing the solid phase-first specific binding partner-analyte-second specific binding partner complex and isolating the solid phase-first specific binding partner-analyte-second specific binding partner complex from the supernatant of the first mixture;

c) removing freely accessible unbound second specific labeled binding partner from the captured solid phase first specific binding partner-analyte-second specific binding partner complex;

d) resuspending the captured solid phase-first specific binding partner-analyte-second specific binding partner complex to form a second mixture comprising resuspended solid phase-first specific binding partner-analyte-second specific binding partner complex;

e) adding a buffer or diluent to the second mixture, wherein said buffer or diluent comprises (i) greater than 0.1% of at least one detergent; (ii) greater than 0.1% of at least one salt; or (iii) a combination of (i) and (ii);

f) incubating the second mixture for a second incubation period;

g) capturing the resuspended solid phase-first specific binding partner-analyte-second specific binding partner complex and isolating the solid phase-first specific binding partner-analyte-second specific binding partner complex from the supernatant of the second mixture;

h) removing residual unbound second specific labeled binding partner from the second mixture; and

i) detecting the labeled second specific binding partner from the first specific binding partner-analyte-second specific binding partner complex in step g) as a measure of the at least one analyte of interest.

In the above immunoassay, the freely accessible unbound second specific labeled binding partner is removed from the first mixture in step c) by washing. Additionally, in the above immunoassay, the residual unbound second specific labeled binding partner is removed from the second mixture in step h) by washing.

In the above immunoassay, step e) further comprises adding a third specific binding partner to the second mixture, wherein said third specific binding partner is labeled with a second detectable label. The second specific binding partner and the third specific binding partner can be the same or can be different. Additionally, the third specific binding partner can comprises multiple binding partners.

In the above immunoassay, the first specific binding partner is an antibody or an antigen. If the first specific binding partner is an antibody, the antibody is selected from the group consisting of a polyclonal antibody, a monoclonal antibody, a chimeric antibody, a human antibody, and an affinity maturated antibody.

In the above immunoassay, the solid phase is selected from the group consisting of a magnetic particle, bead, test tube, microtiter plate, cuvette, membrane, a scaffolding molecule, film, filter paper, disc, and chip.

In the above immunoassay, the first detectable label is selected from the group consisting of a radioactive label, an enzymatic label, a chemiluminescent label, a fluorescent label, a thermometric label, and an immuno-polymerase chain reaction label. The second detectable label is selected from the group consisting of a radioactive label, an enzymatic label, a chemiluminescent label, a fluorescent label, a thermometric label, and an immuno-polymerase chain reaction label. In the above immunoassay, the first detectable label and the second detectable label can be the same or the first detectable label and the second detectable label can be different. In one aspect, the first detectable label and the second detectable label are the same.

In the above immunoassay, the second specific binding partner is immobilized on a solid phase. The solid phase is selected from the group consisting of a magnetic particle, bead, test tube, microtiter plate, cuvette, membrane, a scaffolding molecule, film, filter paper, disc, and chip.

In the above immunoassay, the first incubation period comprises a period of from about 1 minute to about 60 minutes. The second incubation period comprises a period of from about 30 seconds to about 60 minutes.

In the above immunoassay, the immunoassay relates the amount of said first specific binding partner-analyte-second specific binding partner complex in step i) to the amount of the at least one analyte of interest in the test sample either by use of a standard curve for the analyte, or by comparison to a reference standard.

In yet another aspect, the present disclosure relates to a one-step immunoassay comprising the steps of:

a) incubating for a first incubation period a first mixture comprising: (1) a test sample being assessed for at least one analyte of interest, (2) a first specific binding partner that is immobilized on a solid phase, wherein the first specific binding partner binds to the at least one analyte of interest, and (3) a second specific binding partner labeled with a first detectable label, wherein the analyte, first specific binding partner and second specific binding partner form a solid phase-first specific binding partner-analyte-second specific binding partner complex and further wherein said first incubation period comprises a period of from about 1 minute to about 60 minutes;

b) capturing the solid phase-first specific binding partner-analyte-second specific binding partner complex and isolating the solid phase-first specific binding partner-analyte-second specific binding partner complex from the supernatant of the first mixture;

c) removing freely accessible unbound second specific labeled binding partner from the captured solid phase first specific binding partner-analyte-second specific binding partner complex;

d) resuspending the captured solid phase-first specific binding partner-analyte-second specific binding partner complex to form a second mixture comprising resuspended solid phase-first specific binding partner-analyte-second specific binding partner complex;

e) adding a buffer or diluent and a third specific binding partner labeled with a second detectable label to the second mixture, wherein said adding a buffer or diluent comprises (i) greater than 0.1% of at least one detergent; (ii) greater than 0.1% of at least one salt; or (iii) combinations of (i) and (ii);

f) incubating the second mixture for a second incubation period, wherein said second incubation comprises a period of from about 30 seconds to about 60 minutes;

g) capturing the resuspended solid phase-first specific binding partner-analyte-second specific binding partner complex and isolating the solid phase-first specific binding partner-analyte-second specific binding partner complex from the supernatant of the second mixture;

h) removing residual unbound second specific labeled binding partner from the second mixture; and

i) detecting the labeled second specific binding partner from the first specific binding partner-analyte-second specific binding partner complex in step g) as a measure of the at least one analyte of interest.

In the above immunoassay, the freely accessible unbound second specific labeled binding partner is removed from the first mixture in step c) by washing. Additionally, in the above immunoassay, the residual unbound second specific labeled binding partner is removed from the second mixture in step h) by washing.

In the above immunoassay, the first specific binding partner is an antibody or an antigen. If the first specific binding partner is an antibody, the antibody is selected from the group consisting of a polyclonal antibody, a monoclonal antibody, a chimeric antibody, a human antibody, and an affinity maturated antibody.

In the above immunoassay, the solid phase is selected from the group consisting of a magnetic particle, bead, test tube, microtiter plate, cuvette, membrane, a scaffolding molecule, film, filter paper, disc, and chip.

In the above immunoassay, the first detectable label is selected from the group consisting of a radioactive label, an enzymatic label, a chemiluminescent label, a fluorescent label, a thermometric label, and an immuno-polymerase chain reaction label. The second detectable label is selected from the group consisting of a radioactive label, an enzymatic label, a chemiluminescent label, a fluorescent label, a thermometric label, and an immuno-polymerase chain reaction label. In the above immunoassay, the first detectable label and the second detectable label can be the same or the first detectable label and the second detectable label can be different. In one aspect, the first detectable label and the second detectable label are the same.

In the above immunoassay, the second specific binding partner is immobilized on a solid phase. The solid phase is selected from the group consisting of a magnetic particle, bead, test tube, microtiter plate, cuvette, membrane, a scaffolding molecule, film, filter paper, disc, and chip.

In the above immunoassay, the immunoassay relates the amount of said first specific binding partner-analyte-second specific binding partner complex in step i) to the amount of the at least one analyte of interest in the test sample either by use of a standard curve for the analyte, or by comparison to a reference standard.

In the above immunoassay, the buffer or diluent and the third specific binding partner are added sequentially. Alternatively, the buffer or diluent and the third specific binding partner are added simultaneously.

### BRIEF DESCRIPTION OF THE FIGURES

FIG. 1 is a graph showing the concentration of an exemplary analyte of interest, hepatitis B surface antigen (HBsAg, ordinate, pg/mL) vs. the signal/noise ratio (abscissa) in samples tested by a one-step (also referred to herein as the “modified two-step”) assay format of the present disclosure as compared to the traditional two-step assay format of the prior art, as described in Example 1. Symbols: (-♦-) one-step assay format: (-▪-) traditional two-step assay format.

FIGS. 2A and 2B show the reduction in non-specific binding in a hepatitis B surface antigen assay using an optimized diluent described in Example 5.

### DETAILED DESCRIPTION

In general, the present disclosure relates to one-step immunoassays for assessing (e.g., detecting or quantifying) at least one analyte of interest in a test sample. In one aspect, the sensitivity of the one-step immunoassays of the present disclosure are improved from about two to about ten times higher than the sensitivity of a conventional or traditional two-step immunoassays known in the art. In a second aspect, the one-step immunoassays of the present disclosure employ an optimized or improved wash buffer or diluent that contains at least one additive. Immunoassays employing this optimized wash buffer or diluent were found to exhibit improved or increased specificity and sensitivity compared to conventional or traditional two-step immunoassays known in the art.

### A. Definitions

As used herein, the singular forms “a,” “an” and “the” include plural referents unless the context clearly dictates otherwise. For the recitation of numeric ranges herein, each intervening number there between with the same degree of precision is explicitly contemplated. For example, for the range 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9 and 7.0 are explicitly contemplated.

a) Analyte or Analyte of Interest

As used herein, the term “analyte” or “analyte of interest” as used interchangeably herein, generally refers to a substance to be detected. Analytes may include antigenic substances, haptens, antibodies, and combinations thereof. Analytes include, but are not limited to, toxins, organic compounds, DNA, RNA, proteins, peptides, microorganisms, amino acids, nucleic acids, hormones, steroids, vitamins, drugs (including those administered for therapeutic purposes as well as those administered for illicit purposes), drug intermediaries or byproducts, bacteria, virus particles and metabolites of or antibodies to any of the above substances. Specific examples of some analytes include, but are not limited to, brain natriuretic peptide (BNP) 1-32; NT-proBNP; proBNP; preproBNP; troponin I; troponin T; troponin C; antibodies or autoantibodies to cardiovascular antigens, including autoantibodies to any form of troponin; human neutrophil gelatinase-associated lipocalin (hNGAL); VEGF-A, soluble VEGFR-1 (sVEGFR-1, also known as sFlt-1), soluble VEGFR-2 (sVEGFR-2), soluble VEGFR-3 (sVEGFR-3), placenta growth factor (P/GF), tacrolimus; cyclosporine; ferritin; creatinine kinase MB (CK-MB); digoxin; phenytoin; phenobarbitol; carbamazepine; vancomycin; gentamycin; theophylline; valproic acid; quinidine; luteinizing hormone (LH); follicle stimulating hormone (FSH); estradiol, progesterone; C-reactive protein; lipocalins; IgE antibodies; cytokines; vitamin B2 micro-globulin; glycated hemoglobin (Gly. Hb); cortisol; digitoxin; N-acetylprocainamide (NAPA); procainamide; antibodies to rubella, such as rubella-IgG and rubella IgM; antibodies to toxoplasmosis, such as toxoplasmosis IgG (Toxo-IgG) and toxoplasmosis IgM (Toxo-IgM); testosterone; salicylates; acetaminophen; hepatitis B virus surface antigen (HBsAg); antibodies to hepatitis B core antigen, such as anti-hepatitis B core antigen IgG and IgM (Anti-HBC); human immune deficiency virus (HIV); human T-cell leukemia virus (HTLV); hepatitis B e antigen (HBeAg); antibodies to hepatitis B e antigen (Anti-HBe); influenza virus; thyroid stimulating hormone (TSH); thyroxine (T4); total triiodothyronine (Total T3); free triiodothyronine (Free T3); carcinoembryonic antigen (CEA); lipoproteins, cholesterol, and triglycerides; and alpha fetoprotein (AFP). Drugs of abuse and controlled substances include, but are not intended to be limited to, amphetamine; methamphetamine; barbiturates, such as amobarbital, secobarbital, pentobarbital, phenobarbital, and barbital; benzodiazepines, such as propoxy and valium; cannabinoids, such as hashish and marijuana; cocaine; fentanyl; LSD; methaqualone; opiates, such as heroin, morphine, codeine, hydromorphone, hydrocodone, methadone, oxycodone, oxymorphone and opium; phencyclidine; and propoxyphene.

b) Antibody

As used herein, the term “antibody” refers to an immunoglobulin molecule or immunologically active portion thereof, namely, an antigen-binding portion. Examples of immunologically active portions of immunoglobulin molecules include F(ab) and F(ab′)₂ fragments which can be generated by treating an antibody with an enzyme, such as pepsin. Examples of antibodies that can be used in the present disclosure include, but are not limited to, polyclonal antibodies, monoclonal antibodies, chimeric antibodies, human antibodies, humanized antibodies, recombinant antibodies, single-chain Fvs (“scFv”), an affinity maturated antibody, single chain antibodies, single domain antibodies, F(ab) fragments, F(ab') fragments, disulfide-linked Fvs (“sdFv”), and antiidiotypic (“anti-Id”) antibodies and functionally active epitope-binding fragments of any of the above.

c) Detectable Label

In terms of the detectable label, any detectable label known in the art can be used. For example, the detectable label can be a radioactive label (such as, e.g., ³H, ¹²⁵I, ³⁵S, ¹⁴C, ³²P, and ³³P), an enzymatic label (such as, e.g., horseradish peroxidase, alkaline peroxidase, glucose 6-phosphate dehydrogenase, and the like), a chemiluminescent label (such as, e.g., acridinium esters, luminal, isoluminol, thioesters, sulfonamides, phenanthridinium esters, and the like), a fluorescence label (such as, e.g., fluorescein (e.g., 5-fluorescein, 6-carboxyfluorescein, 3′6-carboxyfluorescein, 5(6)-carboxyfluorescein, 6-hexachloro-fluorescein, 6-tetrachlorofluorescein, fluorescein isothiocyanate, and the like)), rhodamine, phycobiliproteins, R-phycoerythrin, quantum dots (e.g., zinc sulfide-capped cadmium selenide), a thermometric label, or an immuno-polymerase chain reaction label. An introduction to labels, labeling procedures and detection of labels is found in Polak and Van Noorden, Introduction to Immunocytochemistry, 2^{nd} ed., Springer Verlag, N.Y. (1997) and in Haugland, Handbook of Fluorescent Probes and Research Chemicals (1996), which is a combined handbook and catalogue published by Molecular Probes, Inc., Eugene, Oreg.

d) Specific Binding Partner

As used herein, the phrase “specific binding partner,” as used herein, is a member of a specific binding pair. That is, two different molecules where one of the molecules, through chemical or physical means, specifically binds to the second molecule. Therefore, in addition to antigen and antibody specific binding pairs of common immunoassays, other specific binding pairs can include biotin and avidin, carbohydrates and lectins, complementary nucleotide sequences, effector and receptor molecules, cofactors and enzymes, enzyme inhibitors, and enzymes and the like. Furthermore, specific binding pairs can include members that are analogs of the original specific binding members, for example, an analyte-analog. Immunoreactive specific binding members include antigens, antigen fragments, antibodies and antibody fragments, both monoclonal and polyclonal and complexes thereof, including those formed by recombinant DNA molecules.

e) Test Sample

As used herein, the term “test sample” generally refers to a biological material suspected of containing and/or being tested for an analyte of interest. The test sample may be derived from any biological source, such as, a physiological fluid, including, but not limited to, whole blood, serum, plasma, interstitial fluid, saliva, ocular lens fluid, cerebral spinal fluid, sweat, urine, milk, ascites fluid, mucous, nasal fluid, sputum, synovial fluid, peritoneal fluid, vaginal fluid, menses, amniotic fluid, semen and so forth. Besides physiological fluids, other liquid samples may be used such as water, food products, and so forth, for the performance of environmental or food production assays. In addition, a solid material suspected of containing the analyte may be used as the test sample. The test sample may be used directly as obtained from the biological source or following a pretreatment to modify the character of the sample. For example, such pretreatment may include preparing plasma from blood, diluting viscous fluids and so forth. Methods of pretreatment may also involve filtration, precipitation, dilution, distillation, mixing, concentration, inactivation of interfering components, the addition of reagents, lysing, etc. Moreover, it may also be beneficial to modify a solid test sample to form a liquid medium or to release the analyte.

### B. One-Step Immunoassays With Improved Sensitivity

In one aspect, the present disclosure relates to immunoassays for assessing (e.g., detecting or quantifying) at least one analyte of interest in a test sample, where the sensitivity of the immunoassay is improved relative to, specifically is from about three to about fifteen times, especially, from about two to about ten times, higher than, the sensitivity of conventional two-step immunoassays known in the art. In addition, the immunoassay of the present disclosure maintains low negative sample background signal (less than about 200 relative light units (“RLU”)).

In a so-called “conventional” or “traditional two-step immunoassay” (further distinguished in the Examples), a mixture containing a test sample being assessed for at least one analyte of interest and a first specific binding partner that is immobilized on a solid phase are incubated for a first incubation period. This first incubation period is for a period of from about 1 to about 20, especially about 18 minutes. After this first incubation period, the mixture is washed and then a second specific binding partner labeled with a first detectable label is added to the mixture to form a solid-phase-first specific binding partner-analyte-second specific binding partner complex. After the addition of the second specific binding partner, the mixture is incubated for a second incubation period. This second incubation period can be for a period of about from 4 to about 20 minutes. After this second incubation period, the mixture is washed and the labeled second specific binding partner from the solid-phase first specific binding partner-analyte-second specific binding partner complex is detected.

In contrast, the immunoassays of the present disclosure are considered to be “one-step” immunoassays. Specifically, and as will be described in more detail herein, in the immunoassays of the present disclosure, a second specific binding partner labeled with a first detectable label is added into the mixture during the first incubation period (rather than in the second incubation period in the conventional “two-step” immunoassay), thus resulting in the immunoassay being considered to be a “modified two-step” or more specifically, a “one-step” immunoassay (the phrases “modified two-step immunoassay” and “one-step immunoassay” will be used interchangeably herein).

Generally, as will be described in more detail herein, according to the present disclosure, the sensitivity of the conventional or traditional two-step immunoassay known in the art can be improved or increased by the addition of a second specific binding partner labeled with a first detectable label into the mixture during the first incubation period. By adding a second specific binding partner labeled with a first detectable label to the first mixture and by using the wash process described herein for removing freely accessible and residual unbound second specific labeled binding partner from the first and second mixtures, the ability to detect the analyte of interest (namely, the sensitivity of the assay) can be improved as compared to conventional or traditional two-step immunoassays. Such improvement appears to be due to, and/or is accompanied by an increase in the analyte positive signal, and/or a reduction in the analyte negative signal. Consequently, the improvement is due to, and/or is accompanied by a increase in the signal to noise ratio (S/N). Additionally, another benefit of the present disclosure is the reduction of negative signal.

Accordingly, in one embodiment, the present disclosure relates to incubating, for a first incubation period, a first mixture comprising (1) a test sample being assessed for at least one analyte of interest; (2) a first specific binding partner that is immobilized on a solid phase and binds to the at least one analyte of interest; and (3) a second specific binding partner, wherein the second specific binding partner is labeled with a first detectable label, wherein the analyte, first specific binding partner and second specific binding partner form a solid phase-first specific binding partner-analyte-second specific binding partner complex. The order in which the test sample containing the at least one analyte of interest, the first specific binding partner and the second specific binding partner are added to form the first mixture is not critical. For example, the test sample (containing the at least one analyte of interest), the first specific binding partner and the second specific binding partner can be added sequentially or simultaneously to form the first mixture. Optionally, like the first specific binding partner, the second specific binding partner can also be immobilized on a solid phase. The solid phase used in the immunoassay (for the first specific binding partner and optionally, the second specific binding partner) can be any solid phase known in the art, such as, but not limited to, a magnetic particle, bead, test tube, microtiter plate, cuvette, membrane, a scaffolding molecule, film, filter paper, disc, and chip.

After the first incubation period, the solid phase-first specific binding partner-analyte-second specific binding partner complex is captured using routine techniques known in the art, such as, but not limited to, magnetic capture (if magnetic particles, such as magnetic microparticles are used), filtration, immunocapture, porous capture membrane, flow capture or nanoparticles. Optimally this first capture step (and optionally any subsequent capture step) is reversible. By “reversible” is meant that the capture step does not preclude release from the capture means or agent, followed by resuspension of the complex and, optionally, subsequent recapture.

Next, the solid phase-first specific binding partner-analyte-second specific binding partner complex is isolated from the first mixture, e.g., by separation from the supernatant of the first mixture. After the isolation of the solid phase-first specific binding partner-analyte-second specific binding partner complex from the first mixture (e.g., separation from the first mixture supernatant), then any freely accessible unbound second specific labeled binding partner is removed from first mixture. As used herein, the phrase “freely accessible unbound second specific labeled binding partner” refers to any unbound second specific labeled binding partner that is not or has not been sequestered or blocked by the solid phase and is capable of being removed using routine techniques known in the art, such as, washing. The freely accessible unbound second specific labeled binding partner can be removed from the first mixture using any technique known in the art, such as washing.

After any freely accessible unbound second specific binding partner complex is removed, then the solid phase-first specific binding partner-analyte-second specific binding partner complex is resuspended to form a second mixture. The resuspension of the solid phase-first specific binding partner-analyte-second specific binding partner complex can be performed by placing the solid phase-first specific binding partner-analyte-second specific binding partner complex into a buffer or diluent and then mixing (such as, but not limited to, by vortexing). For example, if the solid phase-first specific binding partner-analyte-second specific binding partner complex is captured by magnetic capture, the complex can be released from the magnetic capture and into (e.g., resuspended in) a buffer or diluent and then vortexed. Preferably, the buffer or diluent has a pH of from about 5 to about 8. More preferably, the buffer or diluent is a specialized wash solution as described in more detail herein. However, any appropriate buffer or diluent can be employed that does not deleteriously impact the improvements over a conventional or traditional two-step assay, as described herein. Along with any other advantages imparted, the resuspension of the solid phase-first specific binding partner-analyte-second specific binding partner complex also serves to allow for the removal of any remaining or residual unbound second specific labeled binding partner from the solid phase first specific binding partner-analyte-second specific binding partner complex. While not wishing to be bound by any theory, it is believed that the resuspension of the solid phase-first specific binding partner-analyte-second specific binding partner complex during the second incubation allows for formation of a different physical configuration or orientation, of the complex, when recaptured, from the second mixture. This allows any previously sequestered or blocked unbound second specific labeled binding partner to become available or amenable for removal using routine techniques known in the art, such as washing.

Optionally, when the solid phase-first specific binding partner-analyte-second specific binding partner complex is resuspended so as to comprise a second mixture, the resuspension can be done directly into, or by combining with, the recovered supernatant of the first mixture (i.e., that recovered following isolation and removal of solid phase-first specific binding partner-analyte-second specific binding partner complex). Furthermore, in one alternate embodiment, a third specific binding partner can be added into or otherwise employed in the second mixture, either with or without resuspension into or combination with the recovered supernatant of the first mixture. Preferably, the third specific binding partner is labeled with a second detectable label and binds the analyte of interest and not to either the first specific binding partner or the second specific binding partner. The use, potential benefits, and advantages of inclusion of such a third specific binding partner, specifically reduction of any prozone phenomena in said immunoassay as compared to an immunoassay in which such third specific binding partner is not added, as well as the nature and recommended amount for inclusion of the third specific binding partner, are described in U.S. Patent Application No. 60/892,295 (incorporated by reference in its entirety for its teachings regarding same).

If a third specific binding partner is used in the immunoassay of the present disclosure, then the second specific binding partner and the third specific binding partner can be the same as each other, or the second specific binding partner and the third specific binding partner can each be different from each other. Moreover, with use of a third specific binding partner that is labeled with a second detectable label, then the first detectable label and the second detectable label can be the same as each other, or the first detectable label and the second detectable label can each be different than each other. In one aspect, the first detectable label and the second detectable label are the same. Moreover, the first specific binding partner, the second specific binding partner, the third specific binding partner, or any combinations thereof can comprise multiple binding partners. For example, the binding partners may comprise a mixture of antibodies at the same or different concentration.

Preferably, the concentration of the third specific binding partner is lower compared to the concentration of the second specific binding partner. In addition, the use of low concentration third specific binding partner results in lower background signal (hence a higher signal to noise ratio), allowing higher sensitivity analyte detection. More preferably, the amount of third specific binding partner used in the immunoassays described herein is from about 1% to about 50% of the amount of the second specific binding partner.

After the second mixture is formed, then the second mixture is incubated for a second incubation period. After the second incubation period, the solid phase-first specific binding partner-analyte-second specific binding partner complex is captured using routine techniques known in the art, such as, but not limited to, such as, but not limited to, magnetic capture (if magnetic particles, such as magnetic microparticles are used), filtration, immunocapture, porous capture membrane, flow capture or nanoparticles. Next, the solid phase-first specific binding partner-analyte-second specific binding partner complex is isolated from the second mixture (i.e., is separated from the supernatant of the second mixture).

After the isolation of the solid phase-first specific binding partner-analyte-second specific binding partner complex from the second mixture, then any residual unbound second specific labeled binding partner is removed from the isolated and captured solid phase-first specific binding partner-analyte-second specific binding partner complex using any technique known in the art, such as washing.

After the second specific labeled binding partner is removed from the second mixture, then the amount of the first specific binding partner-analyte-second specific binding partner complex in the second mixture is determined.

The length of the first and second incubation periods described in the immunoassays herein can vary depending on a variety of factors, including, but not limited to, the identity of specific binding partners. In general, a person of ordinary skill in the art will be able to readily determine the needed length of time, as the incubations are similar as in known immunoassays. In a preferred embodiment, the first incubation period is for a period of time of from about 5 minutes to about 60 minutes, more preferably from about 15 minutes to about 30 minutes. In a preferred embodiment, the second incubation period is for a period of time of from about 30 seconds to about 30 minutes, more preferably form about 1 minute to about 10 minutes.

As mentioned above, after both the first incubation period and the second incubation period, the freely accessible and residual unbound second specific labeled binding partner is removed from the isolated and captured solid phase-first specific binding partner-analyte-second specific binding partner complex. This freely accessible and residual unbound second specific labeled binding partner can be removed by washing with a buffer, a diluent, a salt, a protein, a polymer, an organic solvent or with any combinations thereof. If a diluent or buffer is used for the washing, it is preferred that the buffer or diluent not deleteriously impact the properties of the immunoassays described herein, and/or e.g., have a pH of from between about 5 to about 8. It has been discovered that a further reduction in background signal can be obtained in the immunoassay of the present disclosure if during the washing, the wash buffer described herein in Section C is used.

In the immunoassays of the present disclosure, the amount of the first specific binding partner-analyte-second specific binding partner complex formed is related to the amount of the analyte in the test sample, optionally either by use of a standard curve for the analyte, by comparison to a reference standard, or by other appropriate means. The standard curve can be generated using serial dilutions of analyte of interest of known concentration, by mass spectroscopy, gravimetrically, and/or by other techniques known in the art. Optionally the amount of the analyte in the test sample is quantitated by measuring the amount of the first detectable label. Optionally the amount of the analyte in the test sample is quantitated by measuring the amount of the second detectable label. In one embodiment, the amount of analyte in the test sample is quantitated by measuring the amount of both the first and the second detectable label. Optionally, the label(s) can be employed in other ways, e.g., for monitoring recovery or other parameter following a particular step.

Additionally, in another embodiment, the immunoassay may further comprise an additional step of washing the second mixture after the addition of the third specific binding partner.

In the immunoassays described herein, the first specific binding partner, the second specific binding partner, the third specific binding partner, the first specific binding partner and the second specific binding partner, the first specific binding partner and the third specific binding partner, the second specific binding partner and the third specific binding partner, or the first specific binding partner, the second specific binding partner and the third specific binding partner can be immobilized on a solid phase. The solid phase can be any material known to those of ordinary skill in the art to which the specific binding partners, such as, but not limited to, antibodies or antigens, can be attached. Examples of solid phases that can be used, include, but are not limited to, a test well in a microtiter plate, nitrocellulose, nylon, a bead or microsphere (including a magnetic, paramagnetic, or superparamagnetic bead or microsphere) or a disc (which can be made out of glass, fiberglass, latex, plastic or a paper material), a gel (for example, a gel through which the polypeptides have been run and which is subsequently dried), a scaffolding molecule (such as, but not limited to, bovine serum albumin, DNA or RNA) or a strip, disc or sheet (which can be made out of nitrocellulose, nylon, plastic or paper). Optionally, the first specific binding partner, the second specific binding partner, the third specific binding partner, the first specific binding partner and the second specific binding partner, the first specific binding partner and the third specific binding partner, the second specific binding partner and the third specific binding partner, or the first specific binding partner, the second specific binding partner and the third specific binding partner can be bound to the solid phase by adsorption, by covalent bonding using a chemical coupling agent or by other means known in the art, provided that such binding does not interfere with the ability of any of the specific binding partners (namely, the first specific binding partner, the second specific binding partner, the third specific binding partner, the first specific binding partner and the second specific binding partner, the first specific binding partner and the third specific binding partner, the second specific binding partner and the third specific binding partner, or the first specific binding partner, the second specific binding partner and the third specific binding partner) to bind to the analyte of interest. Moreover, if necessary, the solid phase can be derivatized to allow reactivity with various functional groups on any of the specific binding partners (namely, the first specific binding partner, the second specific binding partner, the third specific binding partner, the first specific binding partner and the second specific binding partner, the first specific binding partner and the third specific binding partner, the second specific binding partner and the third specific binding partner, or the first specific binding partner, the second specific binding partner and the third specific binding partner).

Such derivatization requires the use of certain coupling agents such as, but not limited to, maleic anhydride, N-hydroxysuccinimide and 1-ethyl-3-(3-dimethylaminopropyl)carbodiimide.

### C. One-Step Immunoassays with Improved Sensitivity and Specificity

In another aspect, the present disclosure relates to immunoassays for assessing (e.g., detecting or quantifying) at least one analyte of interest in a test sample, where the sensitivity and specificity of the immunoassay is improved relative to conventional or traditional two-step immunoassays known in the art. Specifically, the one-step immunoassays of the present disclosure exhibit at least one of the following improvements over conventional or traditional two-step immunoassays known in the art: (a) a reduction in the amount of non-specific binding of the analyte of interest during the immunoassay (a reduction in the non-specific binding results in a reduction in the relative light units (“RLUs”) for test samples that are negative for the analyte of interest leading to a reduced number of false positives); (b) a reduction or minimization in the loss of specific binding of the analyte of interest during the immunoassay; or (c) an increase in signal-to-noise values.

As will also be described in more detail herein, the sensitivity and specificity of the one-step immunoassays of the present disclosure can be improved or increased by the addition of an optimized buffer (also referred to herein as a “wash buffer”) or diluent containing at least one additive during the second incubation period.

Accordingly, in one embodiment, the present disclosure relates to incubating, for a first incubation period, a first mixture comprising (1) a test sample being assessed for at least one analyte of interest; (2) a first specific binding partner that is immobilized on a solid phase and binds to the at least one analyte of interest; and (3) a second specific binding partner, wherein the second specific binding partner is labeled with a first detectable label, wherein the analyte, first specific binding partner and second specific binding partner form a solid phase-first specific binding partner-analyte-second specific binding partner complex. The order in which the test sample containing the at least one analyte of interest, the first specific binding partner and the second specific binding partner are added to form the first mixture is not critical. For example, the test sample (containing the at least one analyte of interest), the first specific binding partner and the second specific binding partner can be added sequentially or simultaneously to form the first mixture.

Optionally, like the first specific binding partner, the second specific binding partner can also be immobilized on a solid phase. The solid phase used in the immunoassay (for the first specific binding partner and optionally, the second specific binding partner) can be any solid phase known in the art, such as, but not limited to, a magnetic particle, bead, test tube, microtiter plate, cuvette, membrane, a scaffolding molecule, film, filter paper, disc, and chip.

After the first incubation period, the solid phase-first specific binding partner-analyte-second specific binding partner complex is captured using routine techniques known in the art, such as, but not limited to, magnetic capture (if magnetic particles, such as magnetic microparticles are used), filtration, immunocapture, porous capture membrane, flow capture or nanoparticles. Optimally this first capture step (and optionally any subsequent capture step) is reversible. By “reversible” is meant that the capture step does not preclude release from the capture means or agent, followed by resuspension of the complex and, optionally, subsequent recapture.

Next, the solid phase-first specific binding partner-analyte-second specific binding partner complex is isolated from the first mixture, e.g., by separation from the supernatant of the first mixture. After the isolation of the solid phase-first specific binding partner-analyte-second specific binding partner complex from the first mixture (e.g., separation from the first mixture supernatant), then any freely accessible unbound second specific labeled binding partner is removed from first mixture. As used herein, the phrase “freely accessible unbound second specific labeled binding partner” refers to any unbound second specific labeled binding partner that is not or has not been sequestered or blocked by the solid phase and is capable of being removed using routine techniques known in the art, such as, washing. The freely accessible unbound second specific labeled binding partner can be removed from the first mixture using any technique known in the art, such as washing.

After any freely accessible unbound second specific binding partner complex is removed, then the solid phase-first specific binding partner-analyte-second specific binding partner complex is resuspended to form a second mixture. The resuspension of the solid phase-first specific binding partner-analyte-second specific binding partner complex can be performed by placing the solid phase-first specific binding partner-analyte-second specific binding partner complex into a buffer or diluent and then mixing (such as, but not limited to, by vortexing). For example, if the solid phase-first specific binding partner-analyte-second specific binding partner complex is captured by magnetic capture, the complex can be released from the magnetic capture and into (e.g., resuspended in) a buffer or diluent and then vortexed. Preferably, the buffer or diluent has a pH of from about 5 to about 8. More preferably, the buffer or diluent is a specialized wash solution as described in more detail herein. However, any appropriate buffer or diluent can be employed that does not deleteriously impact the improvements over a conventional or traditional two-step assay, as described herein. Along with any other advantages imparted, the resuspension of the solid phase-first specific binding partner-analyte-second specific binding partner complex also serves to allow for the removal of any remaining or residual unbound second specific labeled binding partner from the solid phase first specific binding partner-analyte-second specific binding partner complex. While not wishing to be bound by any theory, it is believed that the resuspension of the solid phase-first specific binding partner-analyte-second specific binding partner complex during the second incubation allows for formation of a different physical configuration or orientation, of the complex, when recaptured, from the second mixture. This allows any previously sequestered or blocked unbound second specific labeled binding partner to become available or amenable for removal using routine techniques known in the art, such as washing.

Optionally, when the solid phase-first specific binding partner-analyte-second specific binding partner complex is resuspended so as to comprise a second mixture, the resuspension can be done directly into, or by combining with, the recovered supernatant of the first mixture (i.e., that recovered following isolation and removal of solid phase-first specific binding partner-analyte-second specific binding partner complex). Furthermore, in one alternate embodiment, a third specific binding partner can be added into or otherwise employed in the second mixture, either with or without resuspension into or combination with the recovered supernatant of the first mixture. Preferably, the third specific binding partner is labeled with a second detectable label and binds the analyte of interest and not to either the first specific binding partner or the second specific binding partner. The use, potential benefits, and advantages of inclusion of such a third specific binding partner, specifically reduction of any prozone phenomena in said immunoassay as compared to an immunoassay in which such third specific binding partner is not added, as well as the nature and recommended amount for inclusion of the third specific binding partner, are described in U.S. Patent Application No. 60/892,295 (incorporated by reference in its entirety for its teachings regarding same).

If a third specific binding partner is used in the immunoassay of the present disclosure, then the second specific binding partner and the third specific binding partner can be the same as each other, or the second specific binding partner and the third specific binding partner can each be different from each other. Moreover, with use of a third specific binding partner that is labeled with a second detectable label, then the first detectable label and the second detectable label can be the same as each other, or the first detectable label and the second detectable label can each be different than each other. In one aspect, the first detectable label and the second detectable label are the same. Moreover, the first specific binding partner, the second specific binding partner, the third specific binding partner, or any combinations thereof can comprise multiple binding partners. For example, the binding partners may comprise a mixture of antibodies at the same or different concentration.

Preferably, the concentration of the third specific binding partner is lower compared to the concentration of the second specific binding partner. In addition, the use of low concentration of third specific binding partner results in lower background signal (hence a higher signal to noise ratio), allowing higher sensitivity analyte detection. More preferably, the amount of third specific binding partner used in the immunoassays described herein is from about 1% to about 50% of the amount of the second specific binding partner.

After the second mixture is formed, then the second mixture is incubated for a second incubation period. Preferably, a buffer or diluent is added to the second mixture during the second incubation period. It has been discovered that a further reduction in background signal can be obtained in the immunoassay of the present disclosure if such a buffer (also referred to herein as a “wash buffer”) or diluent (having a pH of between 5 to 8) containing at least one additive is used. Specifically, the additive is: (i) greater than about 0.1% (preferably from about 0.5% to about 4.0%, more preferably from about 1.0% to about 2.0%) of at least one detergent; (ii) greater than about 0.1% (preferably between about 1.0% to about 20.0% more preferably from about 2.0% to about 10.0%) of at least one salt; or (iii) combinations of (i) and (ii). Examples of at least one detergent that can be used include, but are not limited to, the detergents listed below in Table A. The inclusion of the wash buffer in the immunoassay of the present disclosure results in a number of improvements when the immunoassay of the present disclosure is compared to conventional immunoassays known in the art. Specifically, the inclusion of the wash buffer results in at least one of the following improvements over conventional immunoassays known in the art: (a) a reduction in the amount of non-specific binding of the analyte of interest during the immunoassay (a reduction in the non-specific binding results in a reduction in the RLUs for test samples that are negative for the analyte of interest leading to a reduced number of false positives); (b) a reduction or minimization in the loss of specific binding of the analyte of interest during the immunoassay; or (c) an increase in signal-to-noise values.

Specifically, using the wash buffer in the one-step immunoassay of the present disclosure improves the signal-to-noise (S/N) values and sensitivity (ng/ml) in a range of about 1.0 to about 2.5, preferably about 1.3 to about 2.1 fold, compared to the traditional two-step immunoassay assays that uses a wash buffer containing PBS or a diluent that does not at least one additive as described above. Moreover, the use of the wash buffer in the one-step immunoassay of the present disclosure was found to improve the specificity by at least about 1.5 fold when compared to a one-step immunoassay using negative controls.

For avoidance of any doubt, the wash buffer used in the immunoassay of the present disclosure is not a reagent that causes detachment of the detectable label from one or more specific binding partners or the label-carrying complex from the solid phase such as the reagents described, for example, in U.S. Pat. No. 7,029,856. Rather, one of the main purposes of wash buffer of the present disclosure is to reduce, remove or minimize non-specific binding of the test sample to the solid phase and to the specific binding partner labeled with a detectable label.

TABLE A

| | Abbrevi- | | |
|---|---|---|---|
| Detergent | ation | Source | Description |
| Dodecyltrimethylammonium | DTAB | Sigma- | Cationic |
| bromide | | Aldrich | |
| | | (Sigma, St. | |
| | | Louis, MO) | |
| Hexadecyltrimethylammonium | CTAB | Sigma | Cationic |
| bromide | | | |
| N-Dodecyl-N,N-dimethyl-3- | SB3-12 | Sigma | Zwitterionic |
| ammonio-1-propanesulfonate | | | |
| 3-[(3- | Chaps | Sigma | Zwitterionic |
| Cholamidopropyl)dimethyl- | | | |
| ammoniou]- | | | |
| 1-propanesulfonate | | | |
| Sodium dodecyl sulfate | SDS | Sigma | Anionic |
| Lithium dodecyl sulfate | LDS | Sigma | Anionic |

Examples of at least one salt that can be used as an additive include, but are not limited to, the salts listed below in Table B.

TABLE B

| Compound | Abbreviation | Source |
|---|---|---|
| Sodium chloride | NaCl | Sigma |
| Poly (vinylsulfonic | PVSA | Sigma |
| acid, sodium salt) | | |
| solution | | |

Certainly, one skilled in the art, using routine techniques known in the art identify other types of detergents and salts, other than those listed in Tables A and B that could be used in the methods of the present disclosure.

In one aspect, the additive, is greater than 0.5% dodecyltrimethylammonium bromide (DTAB). In another aspect, the additive is greater than 0.5% DTAB and 2.0% NaCl. While not wishing to be bound by any theory, the specialized or optimized wash buffer or diluent described herein reduces background signal by removing detectable label that is non-specifically bound to the first specific binding partner-analyte-second specific binding partner complex or the surface of the reaction vessel (e.g., reduces non-specific binding).

After the second incubation period, the solid phase-first specific binding partner-analyte-second specific binding partner complex is captured using routine techniques known in the art, such as, but not limited to, such as, but not limited to, magnetic capture (if magnetic particles, such as magnetic microparticles are used), filtration, immunocapture, porous capture membrane, flow capture or nanoparticles Next, the solid phase-first specific binding partner-analyte-second specific binding partner complex is isolated from the second mixture (i.e., is separated from the supernatant of the second mixture).

After the isolation of the solid phase-first specific binding partner-analyte-second specific binding partner complex from the second mixture, then any residual unbound second specific labeled binding partner is removed from the isolated and captured solid phase-first specific binding partner-analyte-second specific binding partner complex using any technique known in the art, such as washing.

After the second specific labeled binding partner is removed from the second mixture, then the amount of the first specific binding partner-analyte-second specific binding partner complex in the second mixture is determined.

The length of the first and second incubation periods described in the immunoassays herein can vary depending on a variety of factors, including, but not limited to, the identity of specific binding partners. In general, a person of ordinary skill in the art will be able to readily determine the needed length of time, as the incubations are similar as in known immunoassays. In a preferred embodiment, the first incubation period is for a period of time of from about 5 minutes to about 60 minutes, more preferably from about 15 minutes to about 30 minutes. In a preferred embodiment, the second incubation period is for a period of time of from about 30 seconds to about 30 minutes, more preferably form about 1 minute to about 10 minutes.

As mentioned above, after both the first incubation period and the second incubation period, the freely accessible and residual unbound second specific labeled binding partner is removed from the isolated and captured solid phase-first specific binding partner-analyte-second specific binding partner complex. This freely accessible and residual unbound second specific labeled binding partner can be removed by washing with a buffer, a diluent, a salt, a protein, a polymer, an organic solvent or with any combinations thereof. If a diluent or buffer is used for the washing, it is preferred that the buffer or diluent not deleteriously impact the properties of the immunoassays described herein, and/or e.g., have a pH of from between about 5 to about 8.

In the immunoassays of the present disclosure, the amount of the first specific binding partner-analyte-second specific binding partner complex formed is related to the amount of the analyte in the test sample, optionally either by use of a standard curve for the analyte, by comparison to a reference standard, or by other appropriate means. The standard curve can be generated using serial dilutions of analyte of interest of known concentration, by mass spectroscopy, gravimetrically, and/or by other techniques known in the art. Optionally the amount of the analyte in the test sample is quantitated by measuring the amount of the first detectable label. Optionally the amount of the analyte in the test sample is quantitated by measuring the amount of the second detectable label. In one embodiment, the amount of analyte in the test sample is quantitated by measuring the amount of both the first and the second detectable label. Optionally, the label(s) can be employed in other ways, e.g., for monitoring recovery or other parameter following a particular step.

Additionally, in another embodiment, the immunoassay may further comprise an additional step of washing the second mixture after the addition of the third specific binding partner.

In the immunoassays described herein, the first specific binding partner, the second specific binding partner, the third specific binding partner, the first specific binding partner and the second specific binding partner, the first specific binding partner and the third specific binding partner, the second specific binding partner and the third specific binding partner, or the first specific binding partner, the second specific binding partner and the third specific binding partner can be immobilized on a solid phase. The solid phase can be any material known to those of ordinary skill in the art to which the specific binding partners, such as, but not limited to, antibodies or antigens, can be attached. Examples of solid phases that can be used, include, but are not limited to, a test well in a microtiter plate, nitrocellulose, nylon, a bead or microsphere (including a magnetic, paramagnetic, or superparamagnetic bead or microsphere) or a disc (which can be made out of glass, fiberglass, latex, plastic or a paper material), a gel (for example, a gel through which the polypeptides have been run and which is subsequently dried), a scaffolding molecule (such as, but not limited to, bovine serum albumin, DNA or RNA) or a strip, disc or sheet (which can be made out of nitrocellulose, nylon, plastic or paper). Optionally, the first specific binding partner, the second specific binding partner, the third specific binding partner, the first specific binding partner and the second specific binding partner, the first specific binding partner and the third specific binding partner, the second specific binding partner and the third specific binding partner, or the first specific binding partner, the second specific binding partner and the third specific binding partner can be bound to the solid phase by adsorption, by covalent bonding using a chemical coupling agent or by other means known in the art, provided that such binding does not interfere with the ability of any of the specific binding partners (namely, the first specific binding partner, the second specific binding partner, the third specific binding partner, the first specific binding partner and the second specific binding partner, the first specific binding partner and the third specific binding partner, the second specific binding partner and the third specific binding partner, or the first specific binding partner, the second specific binding partner and the third specific binding partner) to bind to the analyte of interest. Moreover, if necessary, the solid phase can be derivatized to allow reactivity with various functional groups on any of the specific binding partners (namely, the first specific binding partner, the second specific binding partner, the third specific binding partner, the first specific binding partner and the second specific binding partner, the first specific binding partner and the third specific binding partner, the second specific binding partner and the third specific binding partner, or the first specific binding partner, the second specific binding partner and the third specific binding partner). Such derivatization requires the use of certain coupling agents such as, but not limited to, maleic anhydride, N-hydroxysuccinimide and 1-ethyl-3-(3-dimethylaminopropyl)carbodiimide.

### D. Adaptations of the Immunoassays of the Present Disclosure

The methods described herein also can be adapted for use in a variety of automated and semi-automated systems (including those wherein the solid phase comprises a microparticle), as described, e.g., in U.S. Pat. Nos. 5,089,424 and 5,006,309, and as, e.g., commercially marketed, e.g., by Abbott Laboratories (Abbott Park, Ill.). Abbott's platforms include but are not limited to, ARCHITECT®, AxSYM®, IMx® (see, e.g., U.S. Pat. No. 5,294,404, which is hereby incorporated by reference in its entirety), PRISM®, EIA (bead), and Quantum™ II instruments, as well as other platforms. Moreover, the disclosure optionally is adaptable for the Abbott Laboratories' commercial Point of Care (i-STAT®; Abbott Laboratories, Abbott Park, Ill.) electrochemical immunoassay system for performing sandwich immunoassays. Immunosensors, and their methods of manufacture and operation in single-use test devices are described, for example in, U.S. Pat. No. 5,063,081, U.S. Patent Application 2003/0170881, U.S. Patent Application 2004/0018577, U.S. Patent Application 2005/0054078, and U.S. Patent Application 2006/0160164, which are incorporated in their entireties by reference for their teachings regarding same.

In particular, with regard to the adaptation of the present autoantibody assay to the I-STAT® system, the following configuration is preferred. A microfabricated silicon chip is manufactured with a pair of gold amperometric working electrodes and a silver-silver chloride reference electrode. On one of the working electrodes, polystyrene beads (0.2 mm diameter) with immobilized capture antibody are adhered to a polymer coating of patterned polyvinyl alcohol over the electrode. This chip is assembled into an I-STAT® cartridge with a fluidics format suitable for immunoassay. On a portion of the wall of the sample holding chamber of the cartridge there is a layer comprising the second detection antibody labeled with alkaline phosphatase (or other label). Within the fluid pouch of the cartridge is an aqueous reagent that includes p-aminophenol phosphate.

In operation, a sample suspected of containing an analyte of interest is added to the holding chamber of the test cartridge and the cartridge is inserted into the I-STAT® reader. After the second antibody (detection antibody) has dissolved into the sample, a pump element within the cartridge forces the sample into a conduit containing the chip. Here it is oscillated to promote formation of the sandwich between the first capture antibody, analyte, and the labeled second detection antibody. In the penultimate step of the assay, fluid is forced out of the pouch and into the conduit to wash the sample off the chip and into a waste chamber. In the final step of the assay, the alkaline phosphatase label reacts with p-aminophenol phosphate to cleave the phosphate group and permit the liberated p-aminophenol to be electrochemically oxidized at the working electrode. Based on the measured current, the reader is able to calculate the amount of analyte in the sample by means of an embedded algorithm and factory-determined calibration curve.

It further goes without saying that the methods and kits as described herein necessarily encompass other reagents and methods for carrying out the immunoassay. For instance, encompassed are various buffers such as are known in the art and/or which can be readily prepared or optimized to be employed, e.g., for washing, as a conjugate diluent, and/or as a calibrator diluent. An exemplary conjugate diluent is ARCHITECT® conjugate diluent (Abbott Laboratories, Abbott Park, Ill.) containing 2-(N-morpholino)ethanesulfonic acid (MES), other salt, protein blockers, antimicrobial and detergent. An exemplary calibrator diluent is ARCHITECT® calibrator diluent (Abbott Laboratories, Abbott Park, Ill.), which comprises a buffer containing MES, other salt, a protein blocker and an antimicrobial.

Furthermore, as previously mentioned, the methods and kits optionally are adapted for use on an automated or semi-automated system. Some of the differences between an automated or semi-automated system as compared to a non-automated system (e.g., ELISA) include the substrate to which the capture antibody is attached (which can impact sandwich formation and analyte reactivity), and the length and timing of the capture, detection and/or any optional wash steps. Whereas a non-automated format such as an ELISA may include a relatively longer incubation time with sample and capture reagent (e.g., about 2 hours) an automated or semi-automated format (e.g., ARCHITECT®) may have a relatively shorter incubation time (e.g., approximately 18 minutes for ARCHITECT®). Similarly, whereas a non-automated format such as an ELISA may incubate a detection antibody such as the conjugate reagent (Pb264) for a relatively longer incubation time (e.g., about 2 hours), an automated or semi-automated format (e.g., ARCHITECT®) may have a relatively shorter incubation time (e.g., approximately 4 minutes for the ARCHITECT®).

The invention will further be illustrated through one or more specific examples. Any such example(s) represent only preferred embodiments of the invention and are not meant to be limiting.

### Example 1

### Improving HBsAg Quantitation Sensitivity By Using a Modified 2-Step Immunoassay Assay's Highly Efficient Method of Removing Residual Unbound Acridinium Labeled Conjugate

In this example, an automated ARCHITECT® System (Abbott Laboratories, Abbott Park, Ill.) was used to perform an immunoassay that would quantitate Hepatitis B Surface Antigen (HBsAg) in specimens that contained a range of concentrations of HBsAg (Abbott Laboratories, Abbott Park, Ill.) in normal human plasma (Abbott Laboratories, Abbott Park, Ill.).

Dilutions of HBsAg Sensitivity Panels (Abbott Laboratories, Abbott Park, Ill.) containing HBsAg, Ay (0.0 ng/mL-0.355 ng/mL) were tested. Testing was performed in reaction vessels (Abbott Laboratories, Abbott Park, Ill.) that are used for individual tests in the automated Abbott ARCHITECT® System. All the described steps took place in the ARCHITECT® instrument.

Identical HBsAg-containing specimens were tested using both the modified two-step immunoassay (also referred to herein as the “one-step”) format of the present disclosure and the convention or traditional two-step immunoassay format, each as further described below.

### A. Modified Two-Step (One-Step) Assay

The modified two-step (or “one-step”) assay format of the present disclosure comprises the steps of adding the sample, magnetic microparticles, and labeled conjugate antibody simultaneously to an individual reaction vessel.

Each HBsAg dilution was dispensed in the amount of 75 μL into the individual reaction vessels. At the same time, 0.10% EDAC-coated magnetic microparticles (50 μL) (Polymer Science, Monticello, Ind.) with anti-HBsAg monoclonal antibodies (Abbott Laboratories, Abbott Park, Ill.), and anti-HBsAg antibodies labeled with acridinium (Abbott Laboratories, Abbott Park, Ill.), 550 ng/mL were dispensed in the amount of 50 μL into the same reaction vessel. The reaction vessel was then vortexed to mix the sample and reactants. Each reaction mixture was incubated for 18 minutes at 37° C.

During this incubation, the HBsAg in the sample was captured by the anti-HBsAg monoclonal antibodies coated onto the magnetic microparticles.

The anti-HBsAg acridinium labeled antibodies also bind to the HBsAg that was captured by the magnetic microparticles. This formed a microparticle-HBsAg-labeled antibody complex.

Upon completion of the 18 minute incubation, the microparticle-HBsAg-labeled antibody complexes were magnetically captured, and immobilized into a pellet, on the side of the reaction vessel. The exterior surface of the immobilized microparticle-HBsAg-labeled antibody complex pellet was then washed by alternately aspirating the liquid from the vessel, and then adding assay kit wash buffer (ARCHITECT® wash buffer, available from Abbott Laboratories, Abbott Park, Ill.) into the reaction vessel (1 mL wash buffer, repeated 4 times). This initiated the process of the removal of the unbound labeled conjugate antibody from the reaction mixture.

The magnetically captured microparticle-HBsAg-labeled antibody complexes formed during the 18 minute incubation remain in the reaction vessel.

During a second incubation of 4 minutes, buffer was then dispensed in the amount of 50 μL to the reaction vessel containing only the microparticle-HBsAg-labeled antibody complexes. This reaction mixture was then vortexed to disperse the microparticle pellet and release any labeled conjugate that had been sequestered within the pellet. This facilitated the later removal of residual unbound labeled conjugate from microparticle-HBsAg-labeled antibody complexes.

The microparticle-HBsAg-labeled antibody complexes were incubated in buffer for 4 minutes at 37° C.

Upon completion of the 4 minute incubation, the microparticle-HBsAg-labeled antibody complexes were again magnetically captured into a pellet. The exterior surface, of the recaptured pellet, was then repeatedly washed with buffer (1 mL wash buffer, repeated 4 times). This process removed any residual unbound labeled anti-HBsAg antibody from the magnetically captured microparticle-HBsAg-labeled antibody complexes.

The magnetically captured microparticle-HBsAg complex pellet was then released.

The acridinium label (Abbott Laboratories, Abbott Park, Ill.) was then triggered to emit light. This was accomplished by adding a low pH (pH 1) buffer containing H₂O₂ (1.32%) (Abbott Laboratories, Abbott Park, Ill.) was dispensed (100 μL) to the microparticle complexes and vortexing. This step released the anti-HBsAg monoclonal antibodies labeled with acridinium (Abbott Laboratories, Abbott Park, Ill.) that had been bound to HBsAg captured by the microparticles.

The magnetic microparticles were then magnetically captured leaving the released acridinium labeled antiHBsAg antibodies in the reaction mixture solution. This was followed by addition (300 μL) of a pH 13 buffer which “triggers” light, relative light units (RLU) production from the acridinium released into the solution.

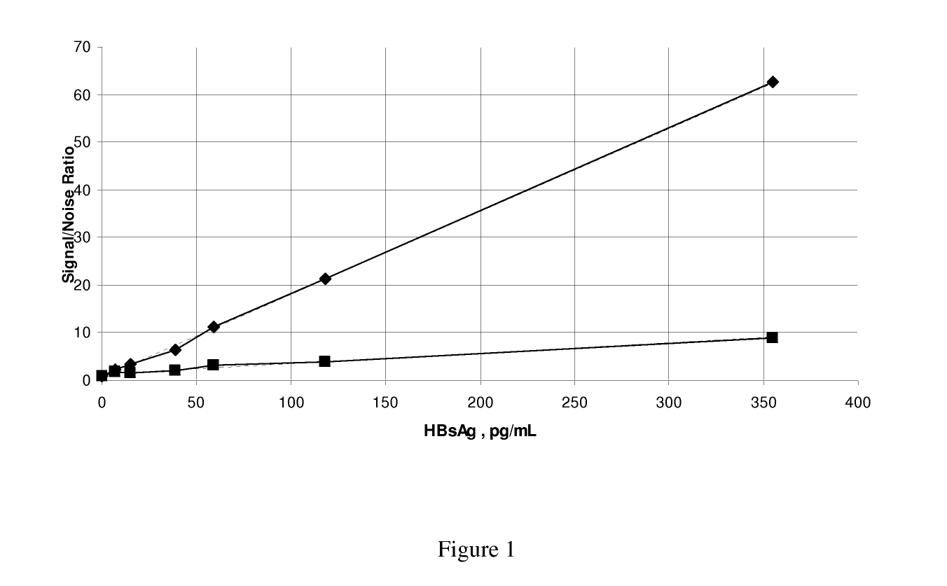

The amount of light that was generated was used to determine the quantity of HBsAg present in the sample (See, Table 1 and FIG. 1).

### B. Traditional or Conventional (Two-Step) Assay

Each HBsAg dilution was dispensed in the amount of 75 μL into individual reaction vessels. At the same time, 0.10% EDAC-coated magnetic microparticles (Polymer Science, Monticello, Ind.) with anti-HBsAg monoclonal antibodies (Abbott Laboratories, Abbott Park, Ill.) were then dispensed, in the amount of 50 μL, into the same reaction vessel. The reaction vessel was then vortexed to mix the sample and magnetic microparticles. Each reaction mixture, was incubated for 18 minutes at 37° C.

During this incubation, the HBsAg in the sample was captured by the anti-HBsAg monoclonal antibodies coated onto the magnetic microparticles. This formed a microparticle-HBsAg complex.

Upon completion of the 18 minute incubation, the microparticle-HBsAg complexes were magnetically captured, and immobilized into a pellet on the side of the reaction vessel. The exterior surface of the immobilized microparticle-HBsAg-labeled antibody complex pellet was then washed by alternately aspirating the liquid from the vessel, and then adding wash buffer into the reaction vessel (1 mL wash buffer, repeated 4 times). This process removed unbound sample the reaction mixture. The magnetically captured microparticle complexes formed during the 18 minute incubation remained in the reaction vessel.

During a second incubation of 4 minutes, the captured magnetic microparticle-HBsAg complex pellet was released from the magnet. Anti-HBsAg antibodies labeled with acridinium, 550 ng/mL (Abbott Laboratories, Abbott Park, Ill.) were then dispensed (50 μL) to the reaction vessel. This reaction mixture was then vortexed to disperse the microparticle pellet and mix the anti-HBsAg antibodies labeled with acridinium. The reaction mixture was incubated for 4 minutes at 37° C.

The anti-HBsAg acridinium labeled antibodies bind to the HBsAg that was captured by the magnetic microparticles. This forms a microparticle-HBsAg-labeled antibody complex.

Upon completion of the 4 minute incubation, the microparticle-HBsAg-labeled antibody complex pellet was magnetically captured again. The pellet exterior surface was then repeatedly washed with buffer (1 mL wash buffer, repeated 4 times). This partially removed unbound labeled anti-HBsAg antibody from the pellet.

The magnetically captured microparticle-HBsAg complex pellet was then released.

The acridinium label (Abbott Laboratories, Abbott Park, Ill.) was then triggered to emit light. This is accomplished by adding a low pH (pH 1) buffer containing H₂O₂ (1.32%) (Abbott Laboratories, Abbott Park, Ill.) was dispensed (100 μL) to the microparticle complexes and vortexing. This step released the anti-HBsAg monoclonal antibodies labeled with acridinium (Abbott Laboratories, Abbott Park, Ill.) that had been bound to HBsAg captured by the microparticles.

The magnetic microparticles were then magnetically captured leaving the released acridinium labeled anti-HBsAg antibodies in the reaction mixture solution. This was followed by addition (300 μL) of a pH 13 buffer which “triggers” light (RLU) production from the acridinium released into the solution.

### Discussion of the Results

The amount of light that was generated in each of the assays as described above was used to determine the quantity of HBsAg present in the sample (See, Table 1 and FIG. 1). In Table 1 below, “Ay” refers to the particular subtype of HBsAg.

TABLE 1

| Two-step format | Mean, | | Modified two-step format | | |
|---|---|---|---|---|---|
| HBsAg Ay, pg/ml | rlu | S/N | HBsAg Ay, pg/ml | Mean, rlu | S/N |
| 0 | 1360 | 1.0 | 0 | 123 | 1.0 |
| 7 | 2617 | 1.9 | 7 | 287 | 2.3 |
| 15 | 2217 | 1.6 | 15 | 428 | 3.5 |
| 39 | 2705 | 2.0 | 39 | 776 | 6.3 |
| 59 | 4315 | 3.2 | 59 | 1374 | 11.2 |
| 118 | 5316 | 3.9 | 118 | 2631 | 21.4 |
| 355 | 12293 | 9.0 | 355 | 7673 | 62.6 |

As shown above in Table 1, the HBsAg signal (RLU) using the modified two-step (or one-step) assay HBsAg signal (RLU) is lower (i.e., 355 pg/mL HBsAg=7673 RLU) compared to the traditional two-step assay HBsAg signal (i.e., 355 pg/mL HBsAg=12293 RLU).

At the same time, the negative specimen (HBsAg 0 pg/mL) signal (123 RLU) of the modified two-step assay is much lower than the traditional two-step assay negative control (1360 RLU). The significantly lower negative results in a significantly higher ratio of HBsAg signal/noise for the modified two-step (or one-step) assay (See, FIG. 1). This in turn translates into more sensitive detection of HBsAg.

Without wishing to be bound by any theory, the improved detection of HBsAg with the modified two-step (or one-step) assay appears to be the result of more efficient removal of unbound acridinium-labeled conjugate from the assay reaction mixture. Adding labeled conjugate to the first assay incubation of the traditional two-step assay format permits highly efficient removal of unbound labeled conjugate. The traditional two-step assay format leaves significant unbound labeled conjugate in the reaction mixture that is triggered to generate light used in HBsAg quantitation.

Adding labeled conjugate to the first assay incubation of a modified two-step (or one-step) assay allows two separate wash sequences, each wash followed by a magnetic microparticle pellet re-suspensions, following labeled conjugate addition to the reaction mixture. The first pellet resuspension goes into the second incubation. This permits removal of any unbound labeled conjugate antibody sequestered inside the magnetically captured pellet.

This is in contrast to the traditional two-step assay in which only a single wash sequence and resuspension takes place after the second incubation during which the labeled conjugate antibody is added to the reaction mixture. During the subsequent wash sequence, unbound labeled conjugate antibody, sequestered inside the magnetically captured pellet, is not exposed to the wash process.

### Example 2

### Inclusion of a Conjugate Wash Buffer Containing a Detergent at 1.0% or greater in a One-Step Immunoassay Improves Detection of Hepatitis B Surface Antigen (HBsAg)

This example demonstrates that the inclusion of a wash buffer with a detergent at 1.0% or greater resulted in improved detection of the target analyte of interest. The assay was performed on an ARCHITECT® instrument per the manufacturer's instructions (Abbott Laboratories, Abbott Park, Ill.). This assay utilized a two-step assay protocol that entails an 18 minute incubation followed by a wash step, a 4 minute incubation followed by a wash step, and a final step that includes triggering and detection of emitted light. Signal or RLUs were reported as measured on the ARCHITECT® instrument.

In this experiment, the sample and both capture and detection reagents were included in the first 18 minute incubation. This format is referred to as a “one-step immunoassay”. In this experiment, the second incubation step or 4 minute incubation was used to wash dispersed microparticles with an optimized wash buffer. In a two-step assay protocol, the conjugate or detection reagent is added in the second step. The addition of a wash buffer deviates from the expected assay format.

Addition of various types of detergents to the wash step that includes dispersion of microparticles in 50 μL of wash buffer, additional mixing and dispersion by adding 150 μL of ARCHITECT® wash buffer (Abbott Laboratories, Abbott Park, Ill.), and a four minute incubation were evaluated. At a concentration of 1.0% or greater, detergents were added to the ARCHITECT® wash buffer (Abbott Laboratories, Abbott Park, Ill.) diluted to a 1× solution and the final solution is referred to herein as a “Conjugate Wash Buffer”. The detergents, SB3-12, CTAB, and Triton X-405 were included in this experiment, and nomenclature and descriptions are found in Table 2, below.

In this experiment, the detergents were added to the 1× ARCHITECT® Wash Buffer at a concentration of 2.0% for SB3-12 and Triton X-405 and at 1.0% for CTAB. These Conjugate Wash Buffers were at pH 6.5. For comparison, phosphate buffered saline (PBS, Abbott Laboratories, Abbott Park, Ill.) at pH 7.2 without detergent or protein additives, the ARCHITECT® 1× Wash Buffer (hereinafter “Arch WB”) at pH 7.0, and the HBsAg Conjugate Diluent (HBsAg CD, −49 plus 200 μg/mL mouse IgG, Abbott Laboratories, Abbott Park, Ill.) at pH 6.3 were used as the wash buffer during the 4 minute incubation step.

The instrument sequence utilizes the ARCHITECT® instrument Two Step 18-4 assay. The instrument sequence was modified to allow for addition of sample, microparticles, and conjugate to the reaction vessel for the first incubation of 18 minutes. All assay steps, volumes of sample and reagents, and incubation times are described in Table 3, below. The microparticles were coated with an anti-HBs monoclonal antibody. The conjugate comprised of acridinium-conjugated goat anti-HBs polyclonal and acridinylated monoclonal antibodies.

The negative control used in this testing was normal human plasma (Abbott Laboratories, Abbott Park, Ill.). The positive sample was prepared by diluting a solution of HBsAg (subtype ay) to 124 pg/mL in normal human plasma. Utilization of HBsAg CD served as a control for an assay format that would typically use this diluent plus conjugated antibody in this 4 minute incubation step.

In Table 4, below, the RLU and signal to noise (S/N) values for the various Conjugate Wash Buffers and other wash buffers are shown. In comparison to testing with HBsAg CD, the S/N values generated with PBS and with the Arch WB were similar. Utilization of the Conjugate Wash Buffer containing the nonionic detergent, Triton X-405, resulted in the lowest S/N value. The Conjugate Wash Buffer containing SB3-12 resulted in a positive RLU value within 10% of the value obtained with the HBsAg CD indicating that inclusion of SB3-12 did not result in loss of specific signal. Using SB3-12 or CTAB in the Conjugate Wash Buffer did result in a decrease in non-specific signal as indicated by the reduced negative control RLU value. Both SB3-12 and CTAB increase the overall signal as compared to the HBsAg CD. Accordingly, optimization of the Conjugate Wash Buffer by inclusion of a detergent, such as SB3-12 or CTAB, in the one-step assay format improved detection of HBsAg.

TABLE 2

| | Abbrevi- | | |
|---|---|---|---|
| Detergent | ation | Source | Description |
| N-Dodecyl-N,N-dimethyl-3- | SB3-12 | Sigma | Zwitterionic |
| ammonio-1-propanesulfonate | | | |
| Hexadecyltrimethylammonium | CTAB | Sigma | Cationic |
| bromide | | | |
| Triton X-405 | TritX405 | Sigma | Nonionic |

TABLE 3

| | | |
|---|---|---|
| Assay Steps | Volume | Time (minutes) |

| | | | |
|---|---|---|---|
| Sample to reaction vessel (rv) | 75 | μl | |
| Conjugate to rv | 50 | μl | |
| Microparticles to rv | 50 | μl | 18 |
| Wash | | | |
| Add ARCHITECT ® 1X Wash | 150 | μl | |
| Buffer to rv | | | |
| Add Conjugate Wash Buffer to rv | 50 | μl | 4 |
| Wash | | | |
| Add Pretrigger to rv | 100 | μl | |
| Add Trigger to rv | 300 | μl | |

TABLE 4

| | | | | Conjugate Wash Buffer | | |
|---|---|---|---|---|---|---|
| | PBS | Arch WB | HBsAg CD | SB3-12 | CTAB | TX405 |
| Negative Control | 254 | 242 | 250 | 164 | 151 | 223 |
| HBsAg Sample (124 pg/ml) | 2250 | 2209 | 2265 | 2097 | 1839 | 1468 |
| HBsAg Sample S/N | 8.9 | 9.1 | 9.1 | 12.8 | 12.2 | 6.6 |
| pH of Conjugate Wash Buffer | 7.2 | 6.8 | 6.3 | 6.5 | 6.5 | 6.5 |

### Example 3

### Inclusion of a Conjugate Wash Buffer with a Combination of Detergent and a Salt Solution in a One-Step Immunoassay Improves Detection of Hepatitis B Surface Antigen (HBsAg)

This example demonstrates that inclusion of a wash buffer with a combination of a detergent and a salt solution resulted in improved detection of the target (e.g., analyte of interest). The general detection method of Example 2 was used to measure the level of HBsAg in samples. The assay configuration to detect HBsAg is described above in Example 2 in Table 3 and was used for all testing in Example 3. As for the reagents, the same antibodies, as used in Example 2, were used to coat microparticles and were acridinylated and used to prepare the detection reagent.

In this experiment, the detergents and salt solution were added to the 1× ARCHITECT® Wash Buffer at a concentration of 2%. The additives are listed in Table 5, below. SB3-12 is a zwitterionic detergent, saponin is a nonionic detergent, and PVSA is an anionic polyelectrolyte. For the Conjugate Wash Buffers containing 2 additives, both additives were added to the 1× ARCHITECT® Wash Buffer at a concentration of 2.0%. The pH of each wash buffer is indicated in Table 6, below. The pH of Conjugate Wash Buffers containing saponin were at the low end of the pH range tested or at pH 4.8, while the Conjugate Wash Buffers containing PVSA were at the high end of the range or at pH 7.5.

As indicated by the RLU values of the HBsAg sample, the pH range of 5 to 7.5 did not have an adverse effect on the acridinium label attached to the detection reagents or on the antigen-antibody binding in the HBsAg assay (See, Table 6). For example, the Conjugate Wash Buffers containing saponin and saponin plus SB3-12 were at the low end of the pH range. The S/N values varied based on the addition of SB3-12 and did not correlate with pH. At the pH range evaluated, the pH of the Conjugate Wash Buffer did not correlate with reduced or enhanced S/N values.

In Table 6, the RLU and signal to noise (S/N) values for the Conjugate Wash Buffers containing PVSA are shown. Inclusion of PVSA to the Conjugate Wash Buffer did not improve the S/N value as compared to SB3-12 alone. When used in combination with SB3-12, there was an improvement in the overall signal. Using SB3-12 with PVSA in the Conjugate Wash Buffer did result in a decrease in non-specific signal as indicated by the reduced negative control RLU value. Accordingly, optimization of the Conjugate Wash Buffer by inclusion of a detergent, such as SB3-12, and a salt solution additive in the one-step assay format improved detection of HBsAg.

TABLE 5

| Additive | Abbreviation | Source |
|---|---|---|
| Poly (vinylsulfonic acid, sodium | PVSA | Sigma |
| salt) solution | | |
| Saponin from quillaja bark | Saponin | Sigma |
| N-Dodecyl-N,N-dimethyl-3- | SB3-12 | Sigma |
| ammonio-1-propanesulfonate | | |

TABLE 6

| | | | SB3-12 | | SB3-12 |
|---|---|---|---|---|---|
| | SB3-12 | Saponin | Saponin | PVSA | PVSA |
| Negative Control | 164 | 273 | 169 | 204 | 150 |
| HBsAg Sample | 2097 | 2328 | 2189 | 2191 | 2131 |
| (124 pg/ml) | | | | | |
| HBsAg Sample S/N | 12.8 | 8.5 | 13.0 | 10.7 | 14.2 |
| pH of Conjugate Wash | 6.5 | 4.8 | 4.8 | 7.5 | 7.5 |
| Buffer | | | | | |

### Example 4

### Detection of Hepatitis B Surface Antigen (HBsAg) with an One-Step Immunoassay that Includes an Optimized Conjugate Wash Buffer Improves the Analytical Sensitivity

This example demonstrates that inclusion of an optimized wash buffer increased the sensitivity of detecting the target analyte of interest. Unless indicated, the general detection method of Example 2 was used to measure the level of HBsAg in samples. The assay configuration used to detect HBsAg is found in Table 7, below and was used for all testing in this Example 4. With respect to reagents, the microparticles used in this testing were coated with two anti-HBs monoclonal antibodies. The conjugate contained the same mixture of antibodies as described in Example 2.

The analytical sensitivity of the assay was determined by testing members of the Abbott HBsAg Sensitivity panel (Abbott Laboratories, Abbott Park, Ill.). This experiment was conducted to gain information on the impact of inclusion of an optimized Conjugate Wash Buffer in an one-step assay format on the analytical sensitivity of the assay. In this experiment, a Conjugate Wash Buffer was prepared by diluting SB3-12 to 2% in the 1× ARCHITECT® Wash Buffer. The HBsAg CD was used for comparison.

The detectable signals measured from the negative control sample were used to calculate a cut-off value (CO) value. The CO values were the mean of the negative control samples multiplied by 3. For the testing with the HBsAg CD, the CO value was 540 (mean=180), and for the Conjugate Wash Buffer prepared with SB3-12 at 2%, the CO value was 375 (mean=125).

The analytical sensitivity values generated in this testing are found in Table 8, below. For both the ay and ad subtypes, the analytical sensitivity values, when the 4 minute incubation step included the Conjugate Wash Buffer with SB3-12, were improved as compared to those generated with the HBsAg CD. For the ad subtype, the analytical sensitivity value, when testing with the Conjugate Wash Buffer containing SB3-12, was improved by greater than 2-fold. The analytical sensitivity was improved by including an optimized Conjugate Wash Buffer.

TABLE 7

| | | |
|---|---|---|
| Assay Steps | Volume | Time (minutes) |

| | | | |
|---|---|---|---|
| Sample to reaction vessel (rv) | 75 | μl | |
| Conjugate to rv | 50 | μl | |
| Microparticles to rv | 50 | μl | 18 |
| Wash | | | |
| Add Conjugate Wash Buffer to rv | 50 | μl | 4 |
| Wash | | | |
| Add Pretrigger | 100 | μl | |
| Add Trigger | 300 | μl | |

TABLE 8

| | HBsAg CD | SB3-12 |
|---|---|---|
| ay ng/ml | 0.022 | 0.016 |
| ad ng/ml | 0.023 | 0.011 |

### Example 5

### Detecting Hepatitis B Surface Antigen (HBsAg) with an One-Step Immunoassay that Includes an Optimized Conjugate Wash Buffer Reduces Non-Specific Binding and Leads to Improved Assay Specificity

This example demonstrates that inclusion of an optimized Conjugate Wash Buffer improved the specificity of detecting the target. Specifically, the general detection method of Example 2 was used to measure the level of HBsAg in samples. The assay configuration to detect HBsAg is found in Example 2 in Table 3 and was used for all testing in this Example 5. As for the reagents, the same antibodies as used in Example 4 were used to coat microparticles and were acridinylated and used to prepare the detection reagent.

In this experiment, a Conjugate Wash Buffer was prepared by diluting SB3-12 to 2% in the 1× ARCHITECT® Wash Buffer and, as in Example 4, the HBsAg CD was used for comparison. Prior to testing for HBsAg in normal donor plasma samples, the samples were thawed and spun at 12,500 rpm for 15 minutes in an Eppendorf 5415C Microfuge (New York, N.Y.) or at 5000 rpm for 43 minutes in an Eppendorf 5804R table top centrifuge (New York, N.Y.). The normal donor plasma panel was comprised of 100 unique samples.

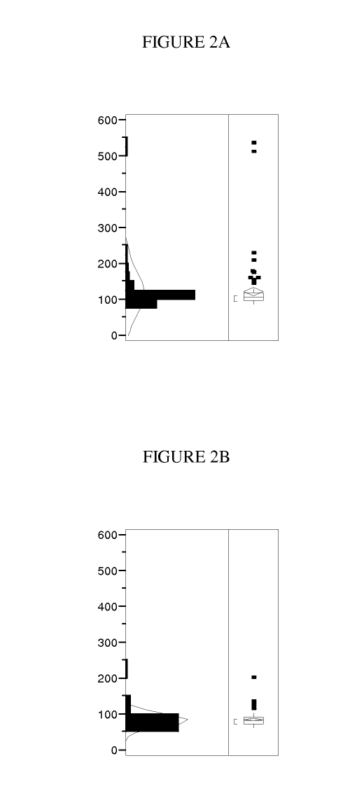

The RLU values of the 100 normal donor plasma samples obtained from testing with the HBsAg CD or the Conjugate Wash Buffer containing SB3-12 at 2% were used to calculate a mean and standard deviation (SD). Using the HBsAg CD, the mean was 120 and the SD was 62.9, and using the Conjugate Wash Buffer containing SB3-12, the mean was 85 and the SD was 18.5. The results of testing the normal donor plasma samples with the two wash buffers are shown in FIGS. 2A and 2B. The inclusion of the Conjugate Wash Buffer with SB3-12 resulted in a tighter distribution of RLU values.

Utilization of the Conjugate Wash Buffer containing SB3-12, a zwitterionic detergent, resulted in a reduction of the mean and SD values of the normal donor plasma samples as compared to the HBsAg CD. When testing with the HBsAg CD, two of the samples had elevated RLU values. These RLU values were 513 and 537. If the CO was calculated by multiplying the mean of the negative control sample by 3, these samples would be considered as initially reactive. Using the same analysis of the results generated with the Conjugate Wash Buffer containing SB3-12, none of the samples tested would be considered reactive. Accordingly, optimization of the Conjugate Wash Buffer by inclusion of a detergent, such as SB3-12, in the one-step assay format improved the specificity of the HBsAg assay.

### Example 6

### Effect of Salt in Conjugate Wash Buffer on Reduction of Non-specific Binding of ARCHITECT® HBsAG Assay

This example compares two conjugate wash buffers of the ARCHITECT® HBsAg assay for reducing non-specific assay background. One conjugate wash buffer is MES buffer pH 6.3 containing 0.5% DTAB without salt and another conjugate wash buffer is MES buffer pH 6.3 containing 0.5% DTAB and 1 M NaCl. These conjugated wash buffers are each referred to herein as a “Conjugate Wash Buffer”.

The ARCHITECT® HBsAg assay is a modified one-step immunoassay for the qualitative detection of HBsAg in human serum and plasma using a chemiluminescent microparticle immunoassay (CMIA) technology with flexible assay protocols, referred to as Chemiflex. Sample, anti-HBs coated paramagnetic microparticles, and acridinium-labeled anti-HBs conjugate are sequentially combined. HBsAg present in the sample and acridinium-labeled anti-HBs conjugate form a sandwich with the anti-HBs coated microparticles in the reaction mixture. After washing, Conjugate Wash buffer is added to the RV and incubated. Following another wash cycle, pre-trigger and trigger solutions are added to the reaction mixture. The resulting chemiluminescent reaction is measured as relative light units (RLUs). A direct relationship exists between the amount of HBsAg in the sample and the RLUs detected by the ARCHITECT® i optical system. The assay configuration used to detect HBsAg is presented in Table 9, below.

TABLE 9

| | | |
|---|---|---|
| Assay Steps | Volume | Time (minutes) |

| | | | |
|---|---|---|---|
| Sample to reaction vessel (rv) | 75 | μl | |
| Conjugate to rv | 50 | μl | |
| Microparticles to rv | 50 | μl | |
| Total reaction mixture in rv | 175 | μl | 18 |
| Wash | | | |
| Add Conjugate Wash Buffer to rv | 50 | μl | 4 |
| Wash | | | |
| Add Pretrigger | 100 | μl | |
| Add Trigger | 300 | μl | |

The data presented in Table 10 indicate that the Conjugate Wash Buffer with 0.5% DTAB with 1 M NaCl salt has higher RLU signal (9258 RLU vs 8672 RLU) of Positive Control and reduces non-specific assay background of 4 normal human plasma (NHP) samples with elevated RLUs (NHP #1: 132 vs 251 RLU, NHP #2: 115 vs 182 RLU, NHP #3: 123 vs 229 RLU, and NHP #4: 156 vs 197 RLU) when compared to 0.5% DTAB Conjugate Wash Buffer without NaCl salt. This result indicates that the 0.5% DTAB Conjugate Wash Buffer with 1 M NaCl salt generates higher signal to noise (S/N) ratio for HBsAg positive sample and lower S/N for HBsAg negative samples.

TABLE 10

| | Conjugate Wash Buffer | | | |
|---|---|---|---|---|
| | | | MES buffer pH 6.3 | |
| | MES buffer pH 6.3 | | with 0.5% DTAB | |
| | with 0.5% DTAB | | and 1 M NaCl | |
| Samples | RLU | S/N | RLU | S/N |
| Negative Control | 95 | | 96 | |
| HBsAg ay, 0.484 ng/ml | 8672 | 91.8 | 9258 | 96.6 |
| NHP #1 | 251 | 2.7 | 132 | 1.4 |
| NHP #2 | 182 | 1.9 | 115 | 1.2 |
| NHP #3 | 229 | 2.4 | 123 | 1.3 |
| NHP #4 | 197 | 2.1 | 156 | 1.6 |

### Example 7

### ARCHITECT® HIV Ag/Ab Combo Assay with an On-Step Assay format that Includes an Optimized Conjugate Wash Buffer Improves Sensitivity

This example compares two conjugate wash buffers in the ARCHITECT® HIV Ag/Ab Combo assay for improving sensitivity of detecting HIV p24 antigen or HIV-1/HIV-2 antibodies. One conjugate wash buffer is MES buffer pH 6.3 containing 0.5% DTAB and 1 M NaCl and another conjugate wash buffer is MES buffer pH 6.3 containing 2% SB3-12 and 0.15 M NaCl. These conjugated wash buffers are each referred to herein as a “Conjugate Wash Buffer”.

The ARCHITECT® HIV Ag/Ab Combo assay is a modified one-step immunoassay to determine the presence of HIV p24 antigen and antibodies to HIV-1 (group M and Group 0) and HIV-2 in human serum and plasma using a CMIA technology with flexible assay protocols, referred to as Chemiflex. Sample, assay diluent, paramagnetic microparticles, and acridinium-labeled conjugates are sequentially combined. HIV p24 antigen and HIV-1/HIV-2 antibodies present in the sample and acridinium-labeled conjugates (HIV-1/HIV-2 antigens (recombinants)), synthetic peptides, and HIV p24 antibody (mouse monoclonal antibody) form a sandwich on HIV-1/HIV-2 antigen and HIV p24 monoclonal (mouse) antibody coated microparticles in the reaction mixture. After washing, the Conjugate Wash Buffers described (See Table 12) below were added to the RV and incubated. Following another wash cycle, pre-trigger and trigger solutions are added to the reaction mixture. The resulting chemiluminescent reaction is measured as RLUs. A direct relationship exists between the amount of HIV antigen and antibodies in the sample and the RLUs detected by the ARCHITECT® i optical system. The assay configuration used to detect HIV p24 antigen or HIV-1/HIV-2 antibodies is presented in Table 11. The presence or absence of HIV p24 antigen and HIV-1/HIV-2 antibodies in the specimen is determined by comparing the chemiluminescent signal in the reaction to the cutoff value (=5*Negative Control RLU value). Specimens with signal to cutoff (S/CO) values greater than or equal to 1.0 are considered reactive for HIV p24 antigen or HIV-1/HIV-2 antibodies. Specimens with signal to cutoff (S/CO) values less than or equal 1.0 are considered non-reactive for HIV p24 antigen or HIV-1/HIV-2 antibodies.

TABLE 11

| | | |
|---|---|---|
| Assay Steps | Volume | Time (minutes) |

| | | | |
|---|---|---|---|
| Sample to reaction vessel (rv) | 100 | μl | |
| Assay Diluent | 50 | μl | |
| Conjugate to rv | 50 | μl | |
| Microparticles to rv | 50 | μl | |
| Total reaction mixture in rv | 250 | μl | 18 |
| Wash | | | |
| Add Conjugate Wash Buffer to rv | 50 | μl | 4 |
| Wash | | | |
| Add Pretrigger | 100 | μl | |
| Add Trigger | 300 | μl | |

The sensitivity improvement of the ARCHITECT® HIV Ag/Ab Combo assay with two Conjugate Wash Buffers was determined by testing ARCHITECT® HIV Ag/Ab Combo Positive Controls (Abbott Laboratories, Abbott Park, Ill.) for HIV p24 antigen and HIV-1/HIV-2 antibodies as samples. Control-1 is a positive control for HIV-1 Group M antibody. Control-2 is a positive control for HIV-2 antibody. Control-3 is a positive control for HIV p24 antigen. Control-4 is a positive control for HIV-1 Group 0 antibody. The RLU and S/CO values for Positive Controls generated in this testing are presented in Table 12. The assay using the Conjugate Wash Buffer with SB3-12 generated lower signal (111 vs 134 RLU) of Negative Control and higher signal for Positive Controls (Control-1: 22153 vs 17732 RLU; Control-2: 6702 vs 5683 RLU; Control-3: 7144 vs 5554 RLU; Control-4: 7327 vs 6686 RLU) as compared to those generated with the Conjugate Wash Buffer containing DTAB. The S/CO values of the Positive Controls with the conjugate wash buffer containing SB3-12 are approximately 1.3-1.5 fold higher than those with the Conjugate Wash Buffer containing DTAB. This result indicates that the ARCHITECT® HIV Ag/Ab Combo assay with the Conjugate Wash Buffer containing SB3-12 is better sensitivity improvement than with the conjugate wash buffer containing DTAB.

TABLE 12

| | Conjugate Wash Buffer | | | |
|---|---|---|---|---|
| | MES buffer with | | MES buffer with | |
| | 0.5% DTAB and | | 2% SB3-12 and | |
| | 1M NaCl | | 0.15M NaCl | |
| Samples | RLU | S/CO | RLU | S/CO |
| Negative Control (NC) | 134 | | 111 | |
| Cutoff RLU value = 5*NC RLU | 670 | | 555 | |
| Control-1 (HIV-1 group M) | 17732 | 26.5 | 22153 | 39.9 |
| Control-2 (HIV-2) | 5683 | 8.5 | 6702 | 12.1 |
| Control-3 (HIV p24 Ag) | 5554 | 8.3 | 7144 | 12.9 |
| Control-4 (HIV-1 Group O) | 6686 | 10.0 | 7327 | 13.2 |

One skilled in the art would readily appreciate that the present disclosure is well adapted to carry out the objects and obtain the ends and advantages mentioned, as well as those inherent therein. The molecular complexes and the methods, procedures, treatments, molecules, specific compounds described herein are presently representative of preferred embodiments, are exemplary, and are not intended as limitations on the scope of the invention. It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present disclosure has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. An immunoassay comprising the steps of:
a) incubating for a first incubation period a first mixture comprising: (1) a test sample being assessed for at least one analyte of interest, (2) a first specific binding partner that is immobilized on a solid phase, wherein the first specific binding partner binds to the at least one analyte of interest, and (3) a second specific binding partner labeled with a first detectable label, wherein the analyte, first specific binding partner and second specific binding partner form a solid phase-first specific binding partner-analyte-second specific binding partner complex;
b) capturing the solid phase-first specific binding partner-analyte-second specific binding partner complex and isolating the solid phase-first specific binding partner-analyte-second specific binding partner complex from the supernatant of the first mixture;
c) removing freely accessible unbound second specific labeled binding partner from the captured solid phase first specific binding partner-analyte-second specific binding partner complex;
d) resuspending the captured solid phase-first specific binding partner-analyte-second specific binding partner complex to form a second mixture comprising resuspended solid phase-first specific binding partner-analyte-second specific binding partner complex;
e) incubating the second mixture for a second incubation period;
f) capturing the resuspended solid phase-first specific binding partner-analyte-second specific binding partner complex and isolating the solid phase-first specific binding partner-analyte-second specific binding partner complex from the supernatant of the second mixture;
g) removing residual unbound second specific labeled binding partner from the second mixture; and
h) detecting the labeled second specific binding partner from the first specific binding partner-analyte-second specific binding partner complex in step g) as a measure of the at least one analyte of interest.

2. The immunoassay of claim 1, wherein the freely accessible unbound second specific labeled binding partner is removed from the first mixture in step c) by washing.

3. The immunoassay of claim 2, wherein the captured solid-phase first specific binding partner-analyte-second specific binding partner complex is washed with a buffer or diluent, a salt, a protein, a polymer, an organic solvent or any combinations thereof.

4. The immunoassay of claim 3, wherein the buffer or diluent contains at least one additive.

5. The immunoassay of claim 1, wherein the residual unbound second specific labeled binding partner is removed from the second mixture in step g) by washing.

6. The immunoassay of claim 1, wherein the second mixture of step d) further comprises a third specific binding partner and the first mixture to form a second mixture, wherein said third specific binding partner is labeled with a second detectable label.

7. The immunoassay of claim 1, wherein said first specific binding partner is an antibody or an antigen.

8. The immunoassay of claim 7, wherein said antibody is selected from the group consisting of a polyclonal antibody, a monoclonal antibody, a chimeric antibody, a human antibody, and an affinity maturated antibody.

9. The immunoassay of claim 1, wherein the solid phase is selected from the group consisting of a magnetic particle, bead, test tube, microtiter plate, cuvette, membrane, a scaffolding molecule, film, filter paper, disc, and chip.

10. The immunoassay of claim 1, wherein the first detectable label is selected from the group consisting of a radioactive label, an enzymatic label, a chemiluminescent label, a fluorescent label, a thermometric label, and an immuno-polymerase chain reaction label.

11. The immunoassay of claim 6, wherein the second detectable label is selected from the group consisting of a radioactive label, an enzymatic label, a chemiluminescent label, a fluorescent label, a thermometric label, and an immuno-polymerase chain reaction label.

12. The immunoassay of claim 6, wherein the first detectable label and the second detectable label are the same.

13. The immunoassay of claim 6, wherein the first detectable label and the second detectable label are different.

14. The immunoassay of claim 1, wherein the second specific binding partner is immobilized on a solid phase.

15. The immunoassay of claim 14, wherein the solid phase is selected from the group consisting of a magnetic particle, bead, test tube, microtiter plate, cuvette, membrane, a scaffolding molecule, film, filter paper, disc, and chip.

16. The immunoassay of claim 6, wherein the second specific binding partner and the third specific binding partner are the same.

17. The immunoassay of claim 6, wherein the second specific binding partner and the third specific binding partner are different.

18. The immunoassay of claim 6, wherein the third specific binding partner comprises multiple binding partners.

19. The immunoassay of claim 1, wherein the first incubation period comprises a period of from about 5 minutes to about 60 minutes.

20. The immunoassay of claim 1, wherein the second incubation period comprises a period of from about 30 seconds to about 30 minutes.

21. The immunoassay of claim 1, wherein the immunoassay relates the amount of said first specific binding partner-analyte-second specific binding partner complex in step g) to the amount of the at least one analyte of interest in the test sample either by use of a standard curve for the analyte, or by comparison to a reference standard.

22. The immunoassay of claim 6, wherein the amount of the at least one analyte of interest in the test sample is quantitated by measuring the amount of the second detectable label.
